(19) United States
(12) Patent Application Publication
(10) Pub. No.: US 2011/0086362 A1
(43) Pub. Date: Apr. 14, 2011

(54) High-Throughput Method for Quantifying Sialylation of Glycoproteins

(75) Inventors: Daniel I. C. Wang, Newton, MA (US); Lam Raga Anggara Markely, Cambridge, MA (US)

(73) Assignee: Massachusetts Institute of Technology, Cambridge, MA (US)

(21) Appl. No.: 12/581,854

(22) Filed: Oct. 19, 2009

Related U.S. Application Data
(60) Provisional application No. 61/250,341, filed on Oct. 9, 2009.

(51) Int. Cl. G01N 33/53

(52) U.S. Cl. 435/7.1

(57) ABSTRACT

Provided herein methods and kits for detecting and/or quantifying sialic acid content of glycosylated molecules that does not require purification of the glycosylated molecule of interest or purification of the labeled product. The methods and kits provided herein are fast and suitable for high-throughput use.

### RELATED APPLICATIONS

This application claims the benefit of priority to U.S. application 61/250,341, filed Oct. 9, 2009, the teachings of which are incorporated herein in their entirety.

### BACKGROUND OF THE TECHNOLOGY

The presence of terminal sialic acid residues affects the properties of various therapeutic glycoproteins in many different ways. One example is recombinant human erythropoietin (rhEPO), which has been successfully used to treat anemia and has 2006 US sales of $10 billion (Aggarwal, 2007). Studies have shown that terminal sialic acid protects rhEPO from clearance by asialoglycoprotein receptors in the liver. As a result, the circulatory half-life of sialo-rhEPO can be as high as 5.6 hours, while that of asialo-rhEPO can be as low as 1.4 minutes (Erbayraktar et al., 2007; Fukuda et al., 1989; Spivak and Hogans, 1989). In addition, the in vivo activity of rhEPO also depends on terminal sialic acid; administration of sialo-rhEPO increases hemoglobin concentration, but that of asialo-rhEPO fails to do so (Erbayraktar et al., 2007). Moreover, the study by Erbayraktar and colleagues (2007) shows that asialo-rhEPO can exert various neuroprotective activities of sialo-rhEPO without increasing the hemoglobin concentration. This finding suggests that asialo-rhEPO can potentially be used for treatment of various neurological diseases. Therefore, engineering sialic acid content is important in defining the therapeutic applications of rhEPO and increasing its circulatory half-life.

Terminal sialic acid also increases anti-inflammatory activities of certain therapeutic recombinant monoclonal antibodies (rMAbs). rMAbs have been widely used to treat cancers and autoimmune diseases, and has US sales of $11.3 billion in 2006 (Aggarwal, 2007). The treatment of cancers utilizes pro-inflammatory activity of certain types of rMAbs while that of autoimmune diseases uses anti-inflammatory activity of other types of rMAbs. Although the current FDA-approved rMAbs are mostly not sialylated (Jefferis, 2006), a study by Kaneko and colleagues (2006) shows that terminal sialic acid affects the anti-inflammatory activity of immunoglobulin G (IgG). Specifically, they found that sialo-human IgG has greater anti-inflammatory activities than asialo-IgG does in a mouse model of rheumatoid arthritis. Therefore, one may increase the sialic acid content of certain types of IgGs to improve their efficacy in treating autoimmune diseases.

Moreover, terminal sialic acid increases the biological activity of IFN-β. IFN-β has been used to treat multiple sclerosis (MS), hepatitis B, and C (Alam, 1995), and has US sales of approximately $2 billion in 2006 (Aggarwal, 2007). Similar to rhEPO, sialo-IFN-β has longer circulatory half-life than asialo-IFN-β does; the former has a circulatory half-life of 1.4 hours, while the latter 0.8 hours (Kasama et al., 1995). In addition, asialo-IFN-β was found to be more effective than sialo-IFN-β in inhibiting the DNA replication of hepatitis B virus (HBV) in vitro (Eto and Takahashi, 1999; Kasama et al., 1995), and decreasing the number of serum HBV virion in vivo (Eto and Takahashi, 1999). The above examples together with other glycoproteins whose circulatory half-lives are affected by terminal sialic acid (Table 1) show the various roles of terminal sialic acid content on the properties of therapeutic glycoproteins. Therefore, monitoring sialic acid content in the production of therapeutic glycoproteins is very important in optimizing their quality and the outcome of disease treatment.

TABLE 1

| | Circulatory half-life | | | | |
|---|---|---|---|---|---|
| Glycoprotein | Sialylated | | Desialylated | | Reference |
| Acetyl- | 12 | hours | 15.3 | minutes | calculated |
| cholinesterase | | | | | from Saxena |
| | | | | | et al., 1997 |
| α1-antitrypsin | 4.6 | days | 4.1 | minutes | Jones et al., |
| | | | | | 1978 |
| Butyryl- | 14.1 | hours | 11 | minutes | calculated |
| cholinesterase | | | | | from Saxena |
| | | | | | et al., 1997 |
| CTLA4Ig | 85 | hours | 0.9 | hours | Flesher et al., |
| | | | | | 1995 |
| Erythropoietin | 5.6 | hours | 1.4 | minutes | Erbayraktar |
| | | | | | et al., 2007 |
| Follicle Stimulating | ~50 | minutes | ~1 | minute | calculated |
| Hormone | | | | | from Morell |
| | | | | | et al., 1971 |
| Human Chorionic | 25 | minutes | <1 | minute | Van Hall |
| Gonadotropin | | | | | et al., 1971 |
| Human Factor VIII | 4 | hours | 5 | minutes | Sodetz et al., |
| | | | | | 1977 |
| Human Luteinizing | 1 | hour | 8.6 | minutes | Burgon et al., |
| Hormone | | | | | 1996 |
| Interferon-β | 1.4 | hours | 0.8 | hours | Kasama et al., |
| | | | | | 1995 |

Various methods have been developed for measuring sialic acid concentration, primarily in human serum (reviewed in Gopaul and Crook, 2006). Existing methods for measuring sialic acid concentration can be classified into four major classes: colorimetric, chromatography, enzymatic, and fluorescence methods. Several examples of colorimetric methods include orcinol method (Klenk and Langerbeins, 1941), resorcinol method (Svennerholm, 1957), thiobarbituric acid assay (TBA) (Warren, 1959), and periodic acid/methyl-3-benzothiazolone-2-hydrazone method (Massamiri et al., 1978). The main disadvantage of current methods is interference by molecules typically found in biological samples such as cell culture samples and clinical samples. For example, hexoses, pentoses, unsaturated fatty acids, DNA, and ATP can interfere with current methods (Gopaul and Crook, 2006, Kuwahara, S. 1990; Nair et al., 2008; and Waters et al., 1992). Therefore, in order to avoid interference by other compounds in an impure sample, existing methods first purify and/or quantify the glycoprotein and the carry out one of the above methods on the purified glycoprotein. However, purification requires additional equipment, and time and is not generally amenable to high-throughput methods.

In addition to colorimetric methods, there are several enzymatic methods for measuring sialic acid concentration (FIG. 1, NANA aldolase: N-acetylneuraminic acid aldolase, AMDH: N-acetylmannosamine dehydrogenase, LDH: lactate dehydrogenase. “Abs, 340 nm” indicates that the sialic acid is measured based on the absorbance of product mixture at 340 nm). The methods on the right primary branch of FIG. 1 correspond to the methods whose end products are derived from pyruvate. The methods on the left primary branch of FIG. 1 correspond to the methods whose end products are derived from N-acetyl-D-mannosamine. Methods whose end products are derived from pyruvate suffer from pyruvate interference and cannot be used for measuring sialic acid content of glycoproteins contained in unpurified samples such as cell culture because the concentration of pyruvate ranges from 1 to 10 mM (estimated from Sigma's Dulbecco's Modified Eagle's Medium). Unless the measurement is accurate up to 6 significant figures, these methods cannot be used for quantifying sialylation of glycosylated molecules such as IFNγ in non-purified form such as cell culture.

On the other hand, methods that measure products derived from N-Acetyl-D-mannosamine require three to five different enzymes. The enzyme used in each step is shown next to the corresponding arrow in FIG. 1. The enzymes must be immobilized or otherwise removed from the reaction. These enzymatic methods are expensive, especially when applied in a large scale.

Several fluorescence methods have also been developed. These methods include derivatization of sialic acid by malononitrile (Honda et al., 1987; Li, 1992), o-phenylenediamine-2HCl (Anumula, 1995), and pyridoxamine (Murayama et al., 1976) to produce fluorescent compounds. The major disadvantage of these methods is interference by, for instance, lipids, 2-deoxy riboses, and ketoacids (Gopaul and Crook, 2006). In particular, pyruvate and glucose found in cell culture samples can interfere with these methods. To minimize the interference, Anumula (1995), Honda et al. (1987), and Li (1992) used these methods together with high performance liquid chromatography (HPLC) for measuring measure sialic acid concentration. Although these methods can minimize the interference, each HPLC can only analyze one sample at a time and is therefore, not high-throughput.

Overall, the existing methods for measuring sialic acid content of glycosylated molecules in non-purified form, such as those produced in cell culture, suffer from interference, costly equipment, and lengthy purification processes. Protein purification may take several hours up to 1 day depending on the sample volume, and it can only handle one sample at a time. Furthermore, sample preparation for High Performance Anion Exchange Chromatography (HPAEC) and Matrix Assisted Laser Desorption Ionization-Time of Flight Mass Spectrometry (MALDI-TOF MS) may take up to 3 days. The processes of purification and sample preparation required by existing methods make these methods incompatible for monitoring sialylation over time such as during batch or fed/batch fermentation or for handling many samples at a time, such as in optimization of culture parameters for increasing sialylation. In addition, protein purification chromatography can cost several hundred thousands of dollars, and the price of MALDI-TOF MS ranges from several hundred thousands of dollars to a million dollars. Furthermore, HPAEC and MALDI-TOF MS cannot be used to provide quantitative measurement.

### SUMMARY

Provided herein are methods and kits for detecting and optionally quantitating sialylation of one or more glycosylated molecules in a biological sample. The methods and kits provided herein for quantifying the terminal sialylation of glycoproteins have several advantages over existing methods. Purification of the glycosylated molecule of interest or purification of the labeled product is not required. In some embodiments, the method uses as little as 60 μL of sample. In some embodiments as little as 3 μM sialic acid can be detected. The methods provided herein are significantly faster than current methods. In contrast to currently available methods, the methods provided herein can take as little as fifteen minutes from the time of obtaining the sample from the biological source (for example harvesting culture supernatant) to determining the quantity of terminal sialic acid residues on the glycosylated molecule of interest. The methods and kits provided herein can be used to analyze multiple samples at the same time, for example as a high throughput assay, are significantly less expensive than current methods, especially in terms of capital cost. The methods and kits provided herein can be used in conjunction with a microplate reader that typically costs around $30,000. In addition, the methods and kits provided herein can be used to quantitatively measure the mole ratio of sialic acid to glycosylated molecule.

In one embodiment, methods of detecting and optionally quantitating sialylation of one or more glycosylated molecules in a sample comprises reducing the sample containing or suspected of containing the one or more glycosylated molecules. The sample can be reduced by contacting the sample with a reducing agent. The reduced sample is then contacted with an agent that is capable of removing terminal sialic acid residues from the one or more glycosylated molecules, generating freed sialic acid residues. The freed sialic acid residues are detectably labeled with a labeling agent, and the detectably labeled freed sialic acid residues are detected. In some embodiments, sialylation of the one or more glycosylated molecules is quantified.

In another embodiment, methods for detecting sialylation of one or more glycosylated molecules in a sample comprises providing at least one reagent suitable for detecting and optionally quantifying sialylation of glycosylated molecules as described herein. The provided reagents can be one or more of: a reducing agent, a reagent suitable for removing terminal sialic acid residues from a glycoprotein, and a labeling reagent suitable for detectably labeling free sialic acid residues. In addition, the method can comprise providing instructions for using the one or more provided reagent to detect sialylation of one or more glycosylated molecules in a sample and instructions for optionally quantifying sialylation of one or more glycosylated molecules in a sample.

Kits for detecting sialylation of one or more glycosylated molecules in a sample are also provided. In some embodiments, the kits provided herein comprise one or more reagents suitable for detecting and optionally quantifying sialylation of glycosylated molecules as described herein. The provided reagents can be one or more of: reducing agent, a reagent suitable for removing a terminal sialic acid residue from a glycoprotein, and a labeling reagent suitable for detectably labeling free a sialic acid residue. The kits provided herein include instructions describing how to use the one or more reagents to detect sialylation of one or more glycosylated molecules in a sample and optionally how to quantify sialylation of one or more glycosylated molecules in a sample.

The various embodiments described herein can be complimentary and can be combined or used together in a manner understood by the skilled person in view of the teachings contained herein.

### BRIEF DESCRIPTION OF FIGURES

FIG. 1 shows a flow chart of methods for measuring sialic acid content of glycoproteins (adapted from Marzouk et al., 2007).

FIG. 2 shows a schematic diagram one embodiment of the method for measuring terminal sugar content of glycosylated molecules.

FIG. 3 shows a bar graph of quantification of sialic acid bound to a fetuin standard.

FIG. 4 shows a bar graph of sialic acid content, number of moles of sialic acid bound to 1 mole of protein, of fetuin standard dissolved in culture medium at five different concentrations.

FIG. 5 shows a sialic acid calibration curve generated using the method described herein.

FIG. 6A-D shows the results of monitoring sialylation of recombinant proteins in cell culture.

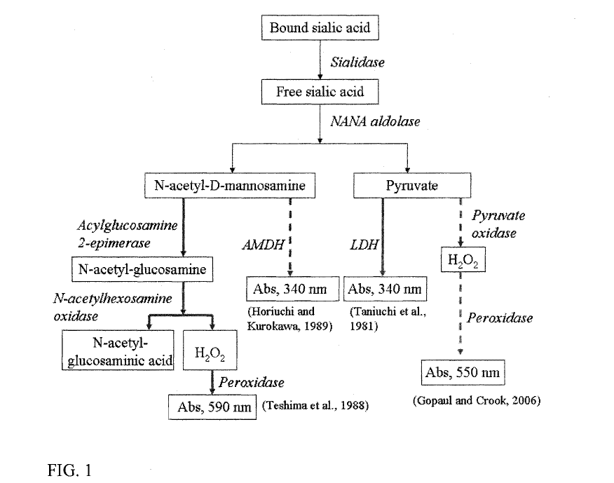

### DETAILED DESCRIPTION

In general, the methods provided herein use chemical and enzymatic reactions to detect and/or quantify the sialylation of glycosylated molecules. The methods provided herein include three steps: reduction of the culture supernatant using a reducing, cleavage of terminal sialic acid from the glycosylated, and derivatization of sialic acid using labeling to produce a detectably labeled sialic acid residue.

In one embodiment, methods of detecting and optionally quantitating sialylation of one or more glycosylated molecules in a sample comprises reducing the sample containing or suspected of containing the one or more glycosylated molecules. The sample can be reduced by contacting the sample with a reducing agent. Potentially interfering molecules in the sample (that is, molecules other than the terminal sialic acid residues present on the glycosylated molecule) that may interact with the labeling agent are rendered non-reactive to the labeling agent. In one embodiment, the interfering molecules are reduced by adding a suitable reducing agent to the sample and incubating under conditions such that the aldehydes, ketones, or other electrophilic groups that may be present on the interfering molecules are reduced.

Suitable reducing agents include those that can reduce carbonyl groups of carbohydrates and that do not break the glycosidic bond that binds the terminal sugar residue to the next to last sugar residue. In one embodiment, a suitable reducing agent is one that does not break the glycosidic bond that binds terminal sialic acid to galactose. Suitable reducing agents include, for example tetrahydroborates [BH4]—such as tetrabutylammonium borohydride, Bu₄N(BH₄), and lithium borohydride. Suitable concentrations of reducing agent can be, for example, 0.05-0.5 M. The reducing agent can be allowed to react with the sample for a time suitable to reduce carbonyl groups of carbohydrates and that do not break the glycosidic bond that binds the terminal sugar residue to the next to last sugar residue. Suitable incubation times can be, for example, 5 to 20 minutes. In one embodiment, the sample is incubated for 5 minutes. The reducing agent can be allowed to react with the sample at a temperature suitable to reduce carbonyl groups of carbohydrates and that do not break the glycosidic bond that binds the terminal sugar residue to the next to last sugar residue. Suitable incubation temperatures can be, for example 4-25°. In one embodiment the sample is incubated at 25° C. In addition, suitable concentrations, incubation times, and incubation temperatures can be determined by one or ordinary skill in the art using routine optimization methods.

The reduced sample is contacted with an agent that is capable of removing terminal sialic acid residues from the one or more glycosylated molecules, generating freed sialic acid residues. Terminal sialic acid residues can be removed using any suitable method that specifically removes the terminal sialic acid without removing other sugar residues that may react with the labeling agent and such that the released or freed sialic acid residue is capable of being detectably labeled with the labeling agent. The sialic acid can be released using chemical or enzymatic means. In some embodiments, an enzyme is used. In some embodiments the enzyme sialidase is used. Enzymes for removing terminal sugar residues are commercially available and can be used according to the manufacturers instructions. In some embodiments, the terminal sialic acid residues are released by incubating the reduced sample/agent mixture at 37° C. for five minutes.

The freed sialic acid residues are detectably labeled with a labeling agent, and the detectably labeled freed sialic acid residues are detected. In some embodiments, sialylation of the one or more glycosylated molecules is quantified. Freed sialic acid residues can be detectably labeled using any agent that is capable of detectably labeling a the freed sugar residue without detectably labeling other types of compounds such as amino acids, proteins, nucleotides, nucleic acid polymers, oligosaccharides, polysaccharides and the like. In some embodiments, any excess or unreacted labeling agent is not be detectable, e.g., is not fluorescent. In some embodiments, if excess or unreacted labeling reagent is fluorescent or otherwise detectable, it is detectable at different excitation or emission wavelength than the labeled freed sugar residue. In some embodiments, the labeling agent is capable of detectably labeling sialic acid residues. In some embodiments, the labeling reagent is malononitrile. Suitable concentrations of labeling agent can be, for example, 0.5 to 15 g/L. In some embodiments, suitable concentrations of labeling reagent can be, for example, at least 7 g/L. The labeling agent can be allowed to react with the sample for a time suitable to detectably label freed sialic acid residues. Suitable incubation times can be, for example, 30 seconds to 30 minutes, depending on the labeling agent used. In some embodiments, suitable incubation times can be, for example, 1-5 minutes. The labeling agent can be allowed to react with freed sialic acid residues at a temperature suitable to detectably label freed sialic acid residues. Suitable incubation temperatures can be, for example, 4-100° C. In addition suitable concentrations, incubation times, and incubation temperatures can be determined by one or ordinary skill in the art using routine optimization methods.

The detectably labeled freed sialic acid residues can be detected using methods compatible with the chosen labeling agent. Colorimetric labeling agents can be detected using a spectrophotometer set at the appropriate wavelength for the colorimetric product. Fluorogenic labeling agents or fluorescent products thereof can be detected using a fluorometer.

In some embodiments of the methods provided herein sialylation of the one or more glycosylated molecules is quantified. In some embodiments, concentration of freed sialic acid residues is determined by measuring fluorescence intensity of the detectably labeled sialic acid residues. The intensity of the signal of the derivatized sialic acid can be used to estimate the concentration of sialic acid released from glycoproteins using the Beer-Lambert law. The concentration can then be divided by the concentration of glycosylated molecule providing the mole ratio of terminal monosaccharides to glycosylated molecule. The concentration of glycosylated molecule in the sample can be determined using standard techniques, such as Enzyme-Linked Immunosorbant Assay (ELISA).

In another embodiment, methods for detecting sialylation of one or more glycosylated molecules in a sample comprises providing at least one reagent suitable for detecting and optionally quantifying sialylation of glycosylated molecules as described herein. The provided reagents can be one or more of: a reducing agent, a reagent suitable for removing terminal sialic acid residues from a glycoprotein, and a labeling reagent suitable for detectably labeling free sialic acid residues. In addition, the method can comprise providing instructions for using the one or more provided reagent to detect sialylation of one or more glycosylated molecules in a sample and instructions for optionally quantifying sialylation of one or more glycosylated molecules in a sample.

As described herein, methods and kits for detecting and/or quantifying sialic acid content of glycosylated molecules are provided. Suitable glycosylated molecules include, for example, glycoproteins, glycolipids, oligosaccharides, and polysaccharides. Suitable glycolipids include, for example, gycophosphatidylinositol (GPI) and gangliosides. As used herein, an oligosaccharide comprises a short chain of carbohydrate structures (or sugar residues) having three to ten repeating units. Suitable oligosaccharides include, for example, sialyl Lewis a (sLe^{a}), sialyl Tn (sTn), sialyl-Lewis x (sLe^{x}), 6-sulpho-sLe^{x}, and sialylated glycans cleaved from glycoproteins or glycolipid by endoglycosidase. As used herein, a polysaccharide comprises a chain of carbohydrate structures having more than ten repeating units. Suitable polysaccharides include, for example, sialic acid-containing meningococcal serogroup B and C polysaccharides, polysialic acid (PSA).

Samples suitable for use in the methods and with the kits provided herein can contain other components in addition to the one or more glycosylated molecules of interest. For example, the sample can contain free sialic acid residues other free sugar molecules, proteins, cells, nucleic acids, and the like. Samples suitable for use in the methods and with the kits provided herein include any fluid or suspension suspected of containing the one or more glycosylated molecules of interest. In some embodiments, the sample is a biological sample. The one or more glycosylated molecules present in the sample can be recombinantly produced, for example by a recombinant host cell.

The sample can be derived from any biological source, such as a physiological fluids (e.g., blood, saliva, sputum, plasma, serum, ocular lens fluid, cerebrospinal fluid, sweat, urine, milk, ascites fluid, synovial fluid, peritoneal fluid, amniotic fluid, cell membrane suspensions, and the like). In addition, the sample can be biopsy material. The sample can be obtained from a human, primate, animal, avian or other suitable source. The sample can also be plant material or cells. The sample can be from prokaryote that has been engineered to produce glycosylated molecules. Suitable plant material can be obtained, for example from tobacco. Suitable plant cells can be, for example tobacco BY2 cells (GT6 cells). Suitable prokaryotes include, for example E. coli.

Samples of tissue or cellular material can be processed into a fluid form suitable for use in the methods and with the kits provided herein. Suitable processing includes homogenization, sonnication, cavitation and the like. The processing can include use of detergents, saponification agents and enzymes to digest or remove molecules such as nucleic acids. The processing can include centrifugation or filtration to remove non-solubilized material. The samples may also be used without any purification or fractionation steps to remove components from the sample, also referred to herein as nonfractionated sample.

In some embodiments, the sample is a bioprocess fluid that contains the one or more glycosylated molecules of interest. For example, the bioprocess fluid can be conditioned culture medium, or milk, blood, plasma, plasma fractions, urine, ascites fluid and the like. In other embodiments, the bioprocess fluid comprises cell homogenates, cell extracts, tissue homogenates, tissue extracts, and the like. The sample can be processed as described herein.

As described herein, recombinant host cells can be any suitable cell that has been altered to generate the one or more glycosylated molecules, for example, by expressing an exogenous gene or nucleic acid that codes for the one or more glycosylated molecules such that the molecule is expressed in glycosylated form. In some embodiments, the recombinant host cell is a fungal cell, insect cell, or mammalian cell into which one or more exogenous nucleic acid sequences have been added. Such nucleic acid sequences include constructs that are capable of expressing, for example, a glycosylated molecule of interest. The exogenous nucleic acid sequence can be introduced into the host cell by, for example, transfection using suitable transfection methods known in the art, or can be introduced by infection using a suitable viral vector, such that the glycosylated molecule encoded by the exogenous nucleic acid is expressed in the host cell. Suitable fungi include, for example, Saccharomyces cerevisiae or Pichia pastoris. Suitable insect cells include, for example, SF9 cells. Recombinant host cells include mammalian cells. Suitable recombinant mammalian cells include, for example, hybridoma cells, or cells into which exogenous nucleic acid has been added as described above. Suitable transfectable cells include, for example, SP2/0 cells, NS/0 cells, Chinese Hamster Ovary (CHO) cells, or Human Embryonic Kidney (HEK). In some embodiments, the bioprocess fluid can contain an endogenous product molecule, for example, clotting factors and immunoglobulin can be isolated from blood or fractions thereof.

Kits

The method for high-throughput quantification of terminal sialylation can be commercialized as a kit. The kit can include one or more reagents suitable for detecting and optionally quantifying sialylation of glycosylated molecules as described herein. The provided reagents can be one or more of: a reducing agent suitable, a reagent suitable for removing a terminal sialic acid residue from a glycoprotein, and a labeling reagent suitable for detectably labeling free a sialic acid residue. The kits provided herein include instructions describing how to use the one or more reagents to detect sialylation of one or more glycosylated molecules in a sample and optionally how to quantify sialylation of one or more glycosylated molecules in a sample. The kit can include one or more multi-well plates, such as 96-well-plates or PCR plates. The kits provided herein can include standard solutions for calibrating the fluorescence intensity for a given sugar residue of interest and labeling agent, and/or reagents to quantify the sialylation of glycoproteins.

In addition to detecting and/or quantifying sialylation of glycosylated molecules, the methods and kits provided herein can be used for various applications, such as monitoring of sialylation during production of therapeutic glycoproteins, and optimization of glycoprotein sialylation by changing various culture condition and supplementation, and analyzing patient samples for alterations in sialylation of glycosylated molecules of interest.

The methods provided herein can be used to quantify other terminal sugar residues such as mannose, galactose, fucose, and N-acetylglucosamine using the appropriate reagent to remove the terminal sugar residue of interest. For example, terminal galactose residues can be removed using the enzyme a-galactosidase or β-galactosidase, terminal fucose residues can be removed using the enzyme a-fucosidase, and terminal N-acetylglucosamine residues can be removed using the enzyme B—N-acetylglucosaminidase. These enzymes are commercially available.

### EXEMPLIFICATION

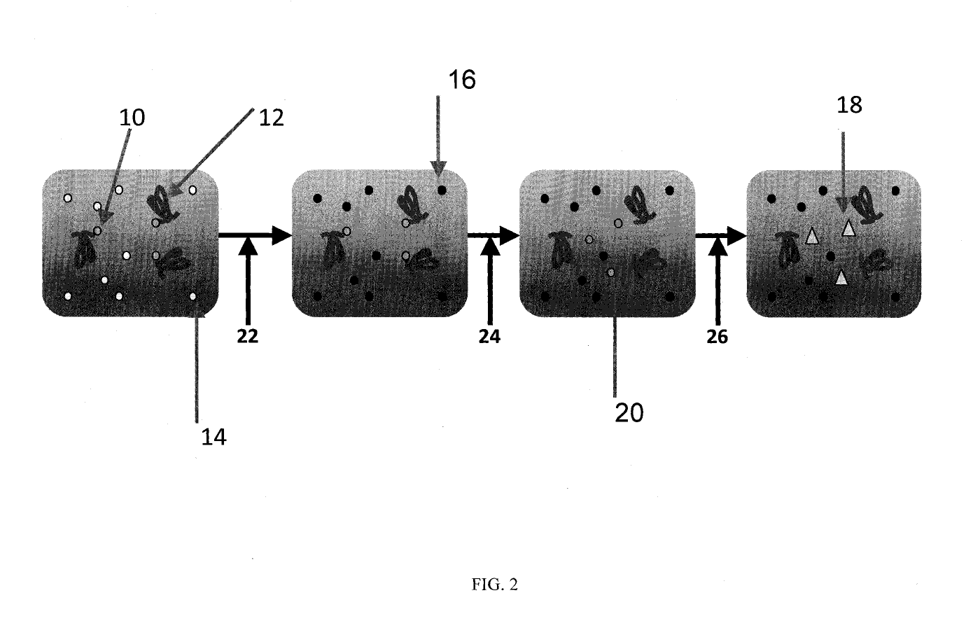

As shown in FIG. 2, in one embodiment of the methods provided herein, the content of a terminal sugar residue (10) of a glycosylated molecule of interest (12) produced in cell culture can be measured. Molecules (14) in cell culture that can interfere with the measurement of the terminal sugar residue of the glycosyalted moledule of interest are reduced using a reducing agent such as Bu₄N(BH₄) (step 22). The reduced molecules (16) will not be able to react with the labeling agent such as malononitrile and therefore, will not become fluorescent molecules. The terminal sugar residues 10 bound to glycosylated molecules 12 are released, for example, sialidase can be used to release terminal sialic acid residues (step 24). The released terminal sugar residues (20) are derivatized with a labeling agent such as malononitrile and borate buffer (step 26) to produce detectably labeled molecules (18). In some embodiments, the intensity of the signal from the detectably labeled molecules can be used to estimate the concentration of terminal sugar residues of interest released from the glycosylated molecule.

### Example 1

### Calibration

Thirty μL of Chinese Hamster Ovary cell (CHO) medium was added to 30 μL of Bu₄N(BH₄) in tetrahydrofuran (THF). Bu₄N(BH4) was dissolved in THF at 0.1M. The mixture was mixed by vortexing for 5 minutes, and then centrifuged for 5 seconds to bring all the liquid down. Thirty μL of a mixture consisting of acetate buffer 1 (0.1 M, pH 5.0) and HCl (1.21 M) at volume ratio 9:1 was added to stop the reaction and the mixture was vortexed for 5 minutes and then centrifuged for 5 seconds to bring all the liquid down.

Two μL of sialic acid standard for calibration (concentration ranging from 0 to 550 μM) and 5 μL acetate buffer 2 (50 mM, pH=5.2) were added and the mixture was heated at 37° C. for 5 minutes and then centrifuged for 5 seconds to bring all the liquid down. The sialic acid concentrations for quantifying sialylation of IFN-γ produced in CHO culture were 0, 5, 10, 20, and 36.7 μM. The sialic acid concentrations for quantifying sialylation of Fetuin were 0, 90, 150, 300, and 550 μM. The concentration of the standard can be adjusted up or down depending on the expected level of sialic acid expected in the glycosylated molecule being tested. Higher concentrations being used with molecules having more sialic acid residues and lower concentrations being used with molecules having fewer sialic acid residues.

Ninety μL of borate buffer (0.15 M, pH=9.4) and 12 μL malononitrile (8 g/L) was added and the mixture was heated at 80° C. for 5 minutes. The reaction was stopped by incubating in an ice bath for ˜1 minute, and then centrifuged for 5 seconds to bring all the liquid down.

Three hundred μL of the mixture from duplicate samples prepared as described above were transferred into a 96-well-plate. Fluorescence intensity at excitation and emission wavelengths of 357 and 430 nm, respectively was measured.

Samples

Thirty μL of sample, e.g. standard glycoprotein dissolved in medium or CHO culture supernatant was added to 30 μL of Bu₄N(BH₄) in THF (0.1 M) and the mixture was mixed by vortexing for 5 minutes, and then centrifuged for 5 seconds to bring all the liquid down. CHO culture supernatant was first centrifuged at 8000 rpm for 10 minutes.

Thirty μL of a mixture consisting of acetate buffer 1 (0.1 M, pH 5.0) and HCl (1.21 M) at volume ratio 9:1 was added to stop the reaction and the mixture was vortexed for 5 minutes and then centrifuged for 5 seconds to bring all the liquid down.

Two μL of water and 5 μL sialidase were added and the mixture was heated at 37° C. for 5 minutes and then centrifuged for 5 seconds to bring all the liquid down. Sialidase was obtained from Roche (10269611001), and the concentrated sialidase stock was diluted 4× with acetate buffer (50 mM, pH=5.2) prior to use.

Ninety μL of borate buffer (0.15 M, pH=9.4) and 12 μL malononitrile (8 g/L) was added and the mixture was heated at 80° C. for 5 minutes. The reaction was stopped by incubating in an ice bath for ˜1 minute, and then centrifuged for 5 seconds to bring all the liquid down.

Three hundred μL of the mixture from duplicate samples prepared as described above were transferred into a 96-well-plate. Fluorescence intensity at excitation and emission wavelengths of 357 and 430 nm, respectively was measured.

Results

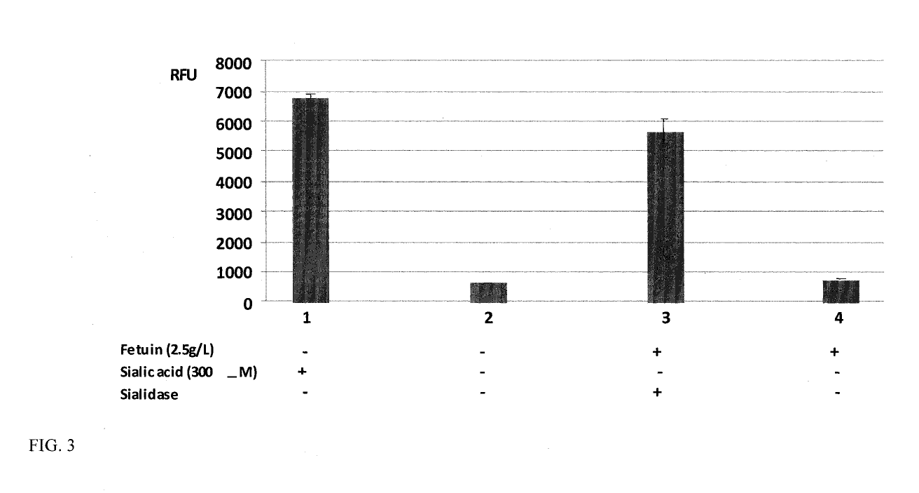

In FIG. 3, fetuin was dissolved in CHO culture medium (50% HyQ PF CHO (Hyclone #30333.01) and 50% CD CHO (Invitrogen #10743029 plus glutamine at 6 mM, pluronic F68 (Invitrogen #24040032) at 1% v/v, methothrexate at 500 nM) at 2.5 g/L. In sample 3, fetuin was dissolved in cell culture medium at 2.5 g/L and the sialic acid content was quantified as described for FIG. 2. Sample 1 is a positive control in which cell culture medium was used without fetuin and 300 μM of sialic acid instead of sialidase was added in the second step in order to mimic the release of sialic acid from fetuin by sialidase. Sample 2 is a negative control in which cell culture medium was used without fetuin and without sialic acid and sialidase in the second step. Sample 4 is another negative control; it is the same as sample 3, but sialidase was not added. The fluorescence intensities indicate that the fluorescence in sample 3 is specific to the sialic acid bound to fetuin. The error bars represent standard errors from 2 independent samples.

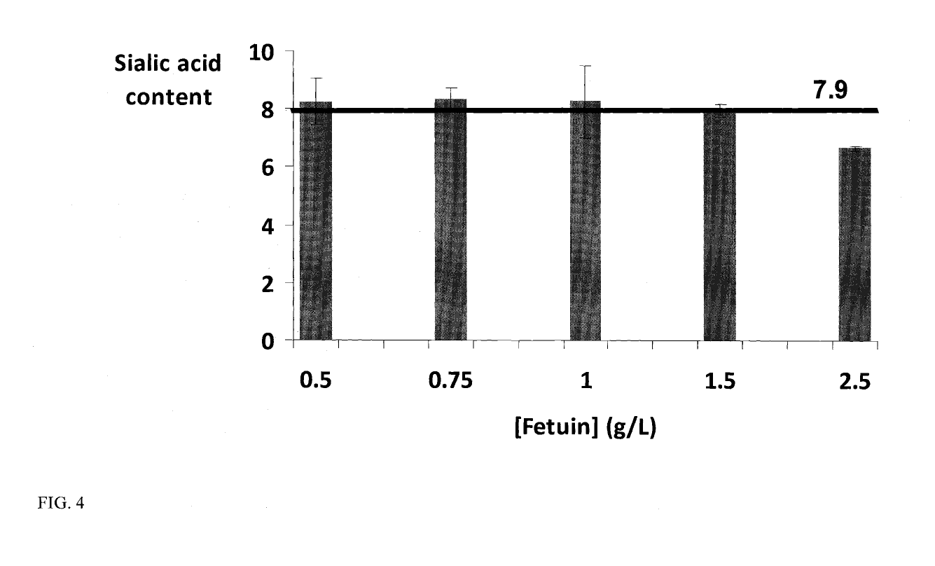

In FIG. 4, the sialic acid content of fetuin standard (Sigma) dissolved in culture medium at 5 different concentrations was measured. The concentration of sialic acid released from fetuin dissolved in culture medium at 5 concentrations was measured by the method provided herein. The sialic acid concentration was then divided by the corresponding concentration of fetuin to calculate sialic acid content. The results were compared with the sialic acid content data provided by Sigma. The sialic acid content measured by the method provided herein and the manufacturer of the fetuin is 7.9±0.3 and 7.6, respectively. Error bars indicate standard errors of 3 independent samples.

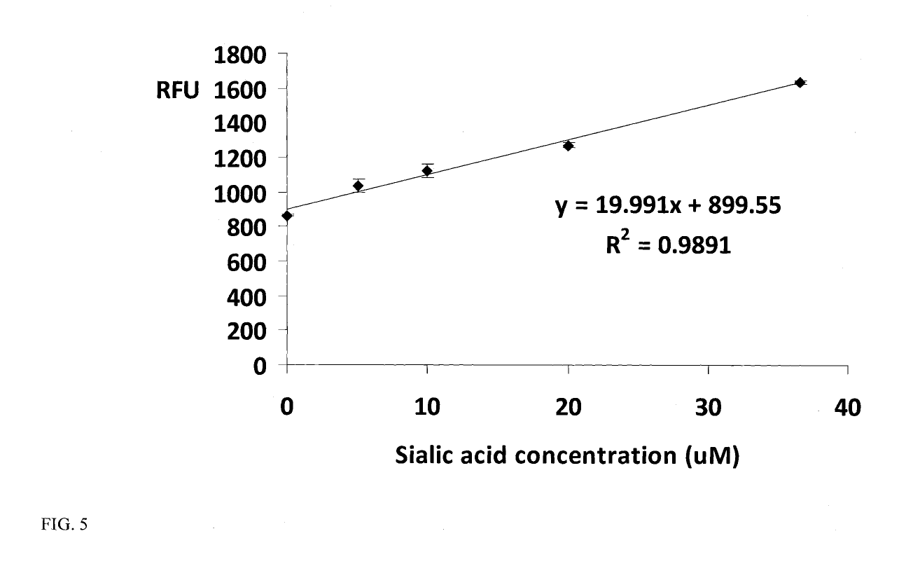

In FIG. 5, a sialic acid calibration curve was generated. CHO culture medium was reduced as described herein. Free sialic acid was added to the reduced culture medium at various concentrations. Malononitrile was then added to convert sialic acid into fluorescent molecules. As little as 2.8 μM sialic acid was detected, as calculated from 10 times standard deviation of the blank. Therefore the method provided herein is at least 10 times more sensitive than commercially available sialic acid quantitation kits provided by Sigma (Sialic-Q) and QA Bio (SialiQuant), both of which have quantitation limit of 33 μM.

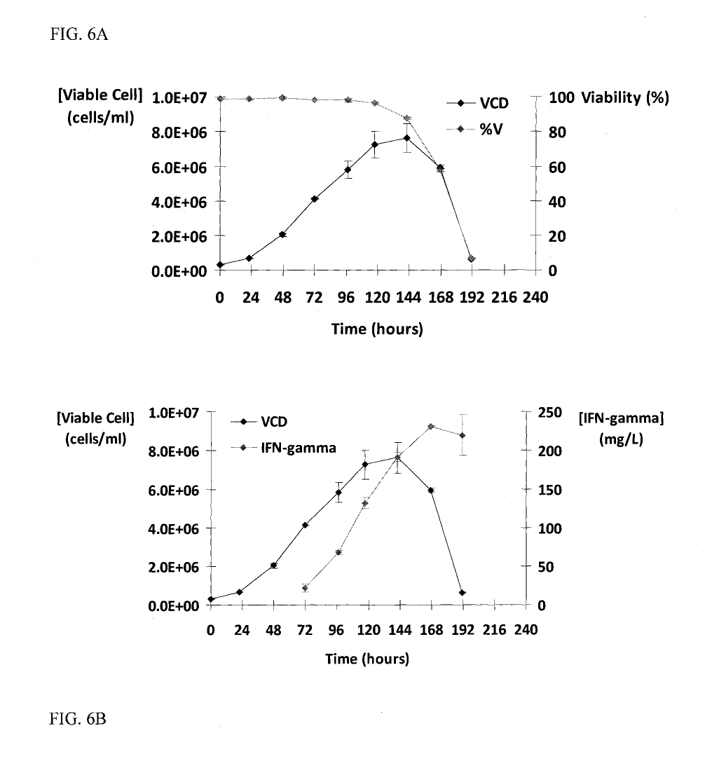

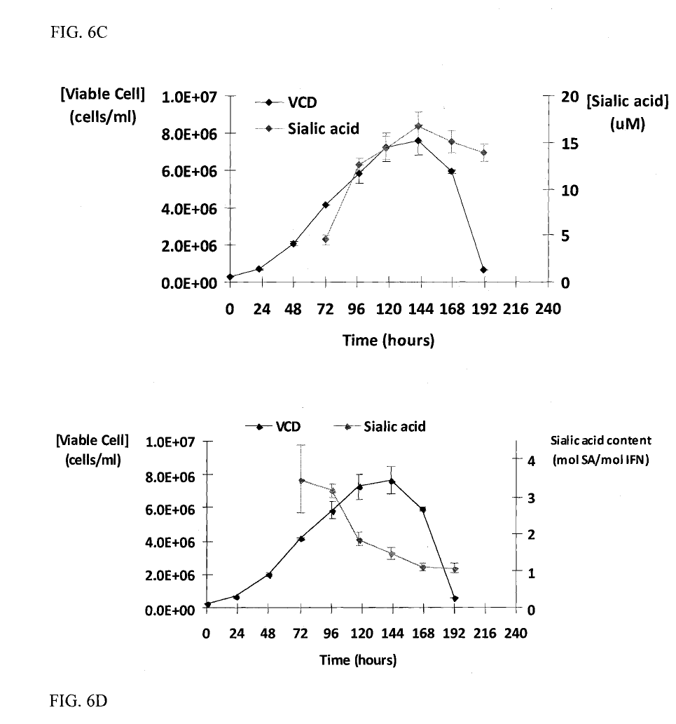

As a proof-of-concept, the methods provided herein have been use to monitor sialylation of recombinant interferon-gamma (IFN-γ) produced in Chinese Hamster Ovary (CHO) cell culture. A high-producing CHO—IFN-γ cell line provided by BTI in Singapore was used. Batch culture of CHO cells that secrete recombinant interferon-γ (IFN-γ) were grown in CHO culture medium at a temperature of 37° C., 90% humidity, 8% CO₂, culture volume of 30 mL, in polycarbonate shake flasks, agitation rate=105 rpm. Culture supernatants were collected every day and analyzed by Enzyme Linked Immunosorbant Assay (ELISA) and the method provided herein to quantify concentration of IFN-γ (FIG. 6B) and total sialic acid bound to IFN-γ (FIG. 6C). The concentration of total sialic acid was divided by IFN-γ concentration to determine the sialic acid content (FIG. 6D). For example, on day 4 (t=97 hours), total sialic acid concentration was found to be 12.7 μM, and IFN-γ concentration was found to be 67.9 mg/L. The molecular weight of IFN-γ is 17 kDa; therefore, the molar concentration of IFN-γ was 3.99 μM. Therefore, sialic acid content was calculated as: 12.7 μM/3.99 μM=3.2. FIG. 6A shows the number of viable cells per milliliter over time. Viable cells were measured by hemacytometer and trypan blue exclusion method.

### Example 2

### Calibration

Twenty eight μL CHO medium was mixed with 15 μL of NaBH₄ in EtOH (0.1 M) and 25 μL EtOH in a PCR plate by vortex for 20 minutes, then centrifuged for 5 seconds to bring all the liquid down.

Thirty two μL of a mixture of Phosphate Buffer (0.1 M, pH=6.0) and HCl (1.21 M) at volume ratio 15:1 was added. The mixture was vortexed and then centrifuged for 5 seconds to bring all the liquid down.

Two μL of sialic acid standard for calibration at desired concentrations and 3.6 μL water were added. The mixture was incubated at 37° C. for 1 hour and then centrifuged for 5 seconds to bring all the liquid down.

Ninety μL borate buffer (0.15 M, pH=9.4) and 12 μL malononitrile (8 g/L) was added and the mixture was incubated at 80° C. for 20 minutes. The mixture was then incubated in ice bath for ˜1 min and then centrifuged for 5 seconds to bring all the liquid down.

One hundred μL of the mixture was transferred into a 96-well-plate. Fluorescence intensity was measured at excitation and emission wavelengths of 357 and 430 nm, respectively.

Sample

Twenty eight μL sample, e.g. standard glycoprotein dissolved in medium or CHO culture supernatant was added to 15 μL of NaBH₄ in EtOH (0.1 M) and 25 μL EtOH in a PCR plate by vortex for 20 minutes, then centrifuged for 5 seconds to bring all the liquid down. CHO culture supernatant was first centrifuged at 8000 rpm for 10 minutes.

Thirty two μL of a mixture of Phosphate Buffer (0.1 M, pH=6.0) and HCl (1.21 M) at volume ratio 15:1 was added. The mixture was vortexed and then centrifuged for 5 seconds to bring all the liquid down.

Two μL of water and 3.6 μL sialidase Roche (11585886001) were added. The mixture was incubated at 37° C. for 1 hour and then centrifuged for 5 seconds to bring all the liquid down. The sialidase was obtained from Roche (11585886001) as a solid. The solid sialidase was dissolved in water at concentration 1 U/20 μL.

Ninety μL borate buffer (0.15 M, pH=9.4) and 12 μL malononitrile (8 g/L) was added and the mixture was incubated at 80° C. for 20 minutes. The mixture was then incubated in ice bath for ˜1 min and then centrifuged for 5 seconds to bring all the liquid down.

One hundred μL of the mixture was transferred into a 96-well-plate. Fluorescence intensity was measured at excitation and emission wavelengths of 357 and 430 nm, respectively.

### REFERENCES

Aggarwal, S. (2007). What's fueling the biotech engine? Nature Biotechnology, 25: 1097-1104.
Alam, J. J. (1995). Interferon-13 treatment of human disease. Current Opinion in Biotechnology, 6: 688-691.
Anumula, K. R. (1995). Rapid quantitative determination of sialic acids in glycoproteins by high-performance liquid chromatography with a sensitive fluorescence detection. Analytical Biochemistry, 230: 24-30.
Burgon, P. G., Stanton, P. G., and Robertson, D. M. (1996). In vivo bioactivities and clearance patterns of highly purified human luteinizing hormone isoforms. Endocrinology, 137: 4827-4836.
Erbayraktar, S., Grasso, G., Sfacteria, A. et al. (2007). Asialoerythropoietin is a nonerythropoietic cytokine with broad neuroprotective activity in vivo. PNAS, 100: 6741-6746.
Eto, T. and Takahashi, H. (1999). Enhanced inhibition of hepatitis B virus production by asialoglycoprotein receptor-directed interferon. Nature Medicine, 5: 577-581.
Flesher, A. R., Marzowski, J., Wang, W. C., and Raff, H. V. (1995). Fluorophore-labeled carbohydrate analysis of immunoglobulin fusion proteins: correlation of oligosaccharide content with in vivo clearance profile. Biotechnology and Bioengineering, 46: 399-407.
Fukuda, M. N., Sasaki, H., Lopez, L., and Fukuda, M. (1989). Survival of recombinant erythropoietin in the circulation: the role of carbohydrates. Blood, 73: 84-89.
Gopaul, K. P. and Crook, M. A. (2006). Sialic acid: A novel marker of cardiovascular disease? Clinical Biochemistry, 39: 667-681.
Honda S., Iwase, S., Suzuki, S., and Kakehi, K. (1987). Fluorometric determination of sialic acids using malononitrile in weakly alkaline media and its application to postcolumn labeling in high-performance liquid chromatography. Anal. Biochem., 160: 455-461.
Jefferis, R. (2006). A sugar switch for anti-inflammatory antibodies. Nature Biotechnology, 24: 1230-1231.
Jones, E. A., Vergalla, J., Steer, C. J., Bradley-Moore, P. R., and Vierling, J. M. (1978). Metabolism of intact and desialylated α₁-antitrypsin. Clinical Science and Molecular Medicine, 55:139-148.
Kaneko, Y., Nimmerjahn, F., and Ravetch, J. V. (2006). Anti-inflammatory activity of immunoglobulin G resulting from Fc sialylation. Science, 213: 670-673.
Kasama, K., Utsumi, J., Matsuo-Ogawa E., et al. (1995). Pharmacokinetics and biologic activities of human native and asialointerferon-βs. Journal of Interferon and Cytokine Research, 15: 407-415.
Klenk, E. and Langerbeins, H. (1941). Orcinol method for measuring sialic acid. Hoppe-Seyler's Z Physiol. Chem., 270: 185-193.
Kuwahara, S. S. (1980). Carbohydrate interference in assays based on the periodate-coupled thiobarbituric acid reagent. Anal. Biochem., 101: 54-60.
Li, K. (1992). Determination of sialic acids in human serum by reversed-phase liquid chromatography with fluorimetric detection. Journal of Chromatography, 579: 209-213.
Massamiri, Y., Beljean, M., Durand, G., Feger, J., Pays, M., and Agneray, J. (1978). Colorimetric assay of sialic acid by methyl-3-benzothiazolinone-2-hydrazone reactant. Anal. Biochem., 91: 618-625.
Morell, A. G., Gregoriadis, G., and Scheinberg, I. H. (1971). The role of sialic acid in determining the survival of glycoproteins in the circulation. The Journal of Biological Chemistry, 246: 1461-1467.
Murayama, J. I., Tomita, M., Tsuji, A., and Hamada, A. (1976). Fluorimetric assay of sialic acids. Analytical Biochemistry, 73: 535-538.
Nair, V., O'Neil, C. L., Wang, P. G. (2008). Malondialdehyde. Encyclopedia of Reagents for Organic Synthesis. Retrieved Feb. 26, 2009 from http://mrw.interscience.wiley.com/eros/articles/rm013/frame.html.
Ngantung, F. A. (2005). Engineering mammalian cell line to improve sialylation. (Doctoral dissertation, Massachusetts Institute of Technology).
Saxena, A., Raveh, L., Ashani, Y., and Doctor, B. P. (1997). Structure of glycan moieties responsible for the extended circulatory life time of fetal bovine serum acetylcholinesterase and equine serum butyrylcholinesterase. Biochemistry, 36: 7481-7489.
Sodetz, J. M., Pizzo, S. V., and McKee, P. A. (1977). Relationship of sialic acid to function and in vivo survival of human factor VIII/von Willebrand factor protein. The Journal of Biological Chemistry, 252: 5538-5546.
Spivak, J. L. and Hogans, B. B. (1989). The in vivo metabolism of recombinant human erythropoietin in the rat. Blood, 73: 90-99.
Svennerholm, L. (1957). Quantitative estimation of sialic acids. Biochim. Biophys. Acta, 24: 604-611.
Van Hall, E. V., Vaitukaitis, J. K., Ross, G. T., Hickman, J. W., and Ashwell, G. (1971). Effects of progressive desialylation on the rate of disappearance of immunoreactive HCG from plasma in rats. Endocrinology, 89: 11-15.
Warren, L. (1959). The thiobarbiturate assay for sialic acids. J. Biol. Chem., 234: 1971-1975.
Waters, P. J., Lewry, E., and Pennock, C. A. (1992). Measurement of sialic acid in serum and urine: clinical applications and limitations. Ann. Clin. Biochem., 29: 625-637.
Wong, N. S. C. (2005). Glycosylation of recombinant protein in Chinese Hamster Ovary (CHO) cells. (Doctoral dissertation, National University of Singapore).

While the technology has been particularly shown and described with reference to specific embodiments, it should be understood by those skilled in the art that various changes in form and detail may be made therein without departing from the spirit and scope of the technology as defined by the appended claims.

What is claimed is:

1. A method for detecting sialylation of one or more glycosylated molecules in a sample comprising;
a) reducing a sample by contacting the sample with a reducing agent, wherein the sample comprises one or more glycosylated molecules;
b) contacting the reduced sample with an agent capable of removing terminal sialic acid residues from the one or more glycosylated molecules, generating freed sialic acid residues;
c) detectably labeling the freed sialic acid residues with a labeling agent, and
d) detecting the detectably labeled freed sialic acid residues and optionally quantifying sialylation of the one or more glycosylated molecules,
thereby detecting sialylation of one or more glycosylated molecules.

2. The method of claim 1, wherein the reducing agent is tetrabutylammonium borohydride (Bu₄N(BH₄)).

3. The method of claim 1, wherein the terminal sialic acid residues are removed by contacting the reduced sample with sialidase.

4. The method of claim 1, wherein the labeling agent is malononitrile.

5. The method of claim 1, wherein the sample comprises cell culture supernatant.

6. The method of claim 1, wherein the sample comprises a clinical sample.

7. The method of claim 1, wherein the sample comprises at least one compound selected from the group consisting of: polyunsaturated fatty acids, DNA, ATP, glucose, fucose, galactose, pyruvate, and mannose.

8. The method of claim 1, wherein the sample and/or the reduced sample is unfractionated.

9. The method of claim 1, wherein detectably labeling the freed sialic acid residues with the labeling agent comprises contacting the reduced sample with the labeling agent.

10. The method of claim 1, wherein the detectably labeled freed sialic acid residues are detected in the presence of the reduced sample or at least one compound selected from the group consisting of: polyunsaturated fatty acids, DNA, ATP, glucose, fucose, galactose, pyruvate, mannose, and the labeling reagent.

11. The method of claim 1, wherein the one or more glycosylated molecules is a glycoprotein.

12. The method of claim 1, wherein the one or more glycosylated molecules is selected from the group consisting of: glycolipid and glycophosphatidylinositol.

13. The method of claim 1, wherein the one or more glycosylated molecules is selected from the group consisting of: oligosaccharides and polysaccharides.

14. The method of claim 1, wherein the one or more glycosylated molecules are selected from the group consisting of an antibody or Fc-containing portion thereof, acetylcholinesterase, α1-antitrypsin, butyrylcholinesterase, CTLA4Ig, erythropoietin, Follicle Stimulating Hormone (FSH), Human Chorionic Gonadotropin (HCG), Human Factor VIII, Human Luteinizing Hormone, and Interferon-β.

15. The method of claim 1, wherein detecting comprises quantifying sialylation of the one or more glycosylated molecules by
e) determining concentration of freed sialic acid residues comprising measuring fluorescence intensity of the detectably labeled sialic acid residues, and
f) dividing the concentration of freed sialic acid residues by a concentration of the one or more glycosylated molecules in the sample.

16. A method for detecting sialylation of one or more glycosylated molecules in a sample comprising;
a) providing at least one reagent selected from the group consisting of: a reducing agent, a reagent suitable for removing terminal sialic acid residues from a glycoprotein, and a labeling reagent suitable for detectably labeling free sialic acid residues, and
b) providing instructions for using the at least one reagent to detect sialylation of one or more glycosylated molecules in a sample and optionally quantify sialylation of one or more glycosylated molecules in a sample.

17. The method of claim 16, wherein the reducing agent is tetrabutylammonium borohydride (Bu₄N(BH₄)).

18. The method of claim 16, wherein the reagent is sialidase.

19. The method of claim 16, wherein the labeling regent is malononitrile.

20. The method of claim 16, wherein the sample comprises cell culture supernatant.

21. The method of claim 16, wherein the sample comprises a clinical sample.

22. The method of claim 16, wherein the sample comprises at least one compound selected from the group consisting of: polyunsaturated fatty acids, DNA, ATP, glucose, fucose, galactose, pyruvate, and mannose.

23. The method of claim 16, wherein the one or more glycosylated molecules is a glycoprotein.

24. The method of claim 16, wherein the one or more glycosylated molecules is selected from the group consisting of: glycolipid and gycophosphatidylinositol (GPI).

25. The method of claim 16, wherein the one or more glycosylated molecules is selected from the group consisting of: oligosaccharides and polysaccharides.

26. The method of claim 25, wherein the one or more glycoprotein are selected from the group consisting of: an antibody or Fc-containing portion thereof, acetylcholinesterase, α1-antitrypsin, butyrylcholinesterase, CTLA4Ig, erythropoietin, Follicle Stimulating Hormone (FSH), Human Chorionic Gonadotropin (HCG), Human Factor VIII, Human Luteinizing Hormone, and Interferon-β.

27. A kit for detecting sialylation of one or more glycosylated molecules in a sample comprising;
a) one or more reagents selected from the group consisting of: a reducing agent, a reagent suitable for removing a terminal sialic acid residue from a glycoprotein, and a labeling reagent suitable for detectably labeling free sialic acid residues, and
b) instructions describing how to use the one or more reagents to detect sialylation of one or more glycosylated molecules in a sample and optionally how to quantify sialylation of one or more glycosylated molecules in a sample.

28. The kit of claim 27, wherein the instructions direct a user to;
i) reduce a sample by contacting the sample with a reducing agent;
ii) remove terminal sialic acid residues from the one or more glycosylated molecules in the reduced sample, generating freed sialic acid residues;
iii) detectably label the freed sialic acid residues, and
iv) detect detectably labeled freed sialic acid residues and optionally quantify sialylation of one or more glycosylated molecules.

29. The kit of claim 27, comprising a reducing agent, wherein the reducing agent is tetrabutylammonium borohydride, Bu₄N(BH₄).

30. The kit of claim 27, comprising a reagent suitable for removing a terminal sialic acid residue from a glycoprotein, wherein the reagent is sialidase.

31. The kit of claim 27, comprising a labeling reagent suitable for detectably labeling free a sialic acid residue, wherein the labeling regent is malononitrile.
